(19) United States
(12) Patent Application Publication
(10) Pub. No.: US 2004/0147762 A1
(43) Pub. Date: Jul. 29, 2004

(54) Asymmetric reductive amination of ketones

(76) Inventor: Xumu Zhang, State College, PA (US)

Correspondence Address: McDERMOTT WILL & EMERY, 600 13th Street, N.W., Washington, DC, 20005-3096, US

(21) Appl. No.: 10/701,081

(22) Filed: Nov. 5, 2003

Related U.S. Application Data
(60) Provisional application No. 60/424,663, filed on Nov. 6, 2002.

(51) Int. Cl. C 07C 2 9/24; C07D 207/00

(52) U.S. Cl. 548/400; 564/398

(57) ABSTRACT

Processes for the preparation of compounds having a chiral carbon substituted with an amine are disclosed. The processes include admixing a ketone with an amine in the presence of a catalyst having a chiral phosphine ligand and an acid. The admixture can also contain a reducing additive. The admixture is then exposed to hydrogen to directly and asymmetrically aminate the ketone.

### CROSS REFERENCE TO RELATED APPLICATION

This application claims priority from Provisional Patent Application No. 60/424,663, filed Nov. 6, 2002, entitled Asymmetric Reductive Amination of Ketones, the entire disclosure of which is hereby incorporated herein by reference.

### FIELD OF THE DISCLOSURE

The present invention relates to asymmetric reductive amination of ketones and in particular to transition metal catalyzed amination of ketones for the production of chiral amines for pharmaceutical and agricultural compounds.

### BACKGROUND

Chiral amines are of paramount significance in pharmaceutical and agrochemical substances. Their formation drives the development of efficient methods for catalytic asymmetric reactions. Most of the past studies in this field, however, have focused on the enantioselective reduction of a C-N double bond. In contrast to the high enantioselectivities observed in asymmetric reduction of both alkenes and ketones (Brown, J. M.; Halterman, R. L.; Ohkuma, T.; Noyori, R. In Comprehensive Asymmetric Catalysis; Jacobsen, E. N., Pfaltz, A., Yamamoto, H., Eds.; Springer: Berlin, 1999; Vol. 1, pp 121-246. (c) Noyori, R.; Ohkuma, T. Angew. Chem. Int. Ed. 2001, 40, 40-73), only limited success has been achieved in the enantioselective hydrogenation of imines (Blaser, H.-U.; Springer, F. In Comprehensive Asymmetric Catalysis; Jacobsen, E. N., Pfaltz, A., Yamamoto, H., Eds.; Springer: Berlin, 1999; Vol. 1, pp 247-265). Among them, a variety of chiral Ti, Ir, Rh, Ru, Pd complexes have been investigated as catalysts for the reduction of imines (Yun, J.; Buchwald, S. L. J. Org. Chem. 2000, 65, 767-774; Xiao, D.; Zhang, X. Angew. Chem. Int. Ed. 2001, 40, 3425-3428; Blaser, H.-U.; Buser, H.-P.; Hausel, R.; Jalett, H.-P.; Spindler, F. J. Organomet. Chem. 2001, 621, 34-38; Murahashi, S.-I.; Tsuji, T.; Ito, S. Chem. Commun. 2000, 409-410; Kainz, S.; Brinkmann, A.; Leitner, W.; Pfaltz, A. J. Am. Chem. Soc. 1999, 121, 6421-6429; Schnider, P.; Koch, G.; Pretot, R.; Wang, G.; Bohnen, F. M.; Kruger, C.; Pfaltz, A. chem. Eur. J. 1997, 3, 887-892; Morimoto, T.; Suzuki, N.; Achiwa, K. Heterocycles 1996, 43, 2557-2560; Morimoto, T.; Nakajima, N.; Achiwa, K. Synlett 1995, 748-750; Tani, K.; Onouchi, J.; Yamagata, T.; Kataoka, Y. Chem. Lett. 1995, 955-956).

Without isolating and purifying the imines, the direct (one-pot) asymmetric reductive amination of ketones or aldehydes with amines is a simple and practical way for the preparation of chiral amines. However, it has not received adequate attention. Only two preliminary studies have been reported (Blaser, H.-U.; Buser, H.-P.; Jalett, H.-P.; Pugin, B.; Spindler, F. Synlett 1999, 867-868; Tararov, V. I.; Kadyrov, R.; Riermeier, T. H.; Borner, A. Chem. Commun. 2000, 1867-1868). The first example of asymmetric direct reductive amination was reported by Blaser et al. Using the Ir-Xyliphos complex, they found that methoxyacetone reacted with 2-methyl-5-ethyl-aniline to yield an enriched chiral amine as a precursor of an important grass herbicide, with complete conversion and 78% enantiomeric excess (ee) (10⁴ turnovers). Borner et al. developed a Rh-chiral diphosphine catalyst for asymmetric reductive amination of α-keto acid derivatives. For example, enantiomerically enriched N-benzyl α-amino acid was obtained in 59% yield and 38% ee. However the corresponding α-hydroxy acid was also generated.

Recently, a chiral ligand, f-Binaphane, was developed which has shown excellent reactivities and enantioselectivities for Ir-catalyzed asymmetric hydrogenation of acyclic imines (up to 99% ee) (Xiao, D.; Zhang, X. Angew. Chem. Int. Ed. 2001, 40, 3425-3428; See also WO 01/14299 to Zhang et al.). In the reference to Zhang et al., WO 01/14299, transition metal complexes and their use in asymmetric synthesis is disclosed including the enantioselective hydrogenation of imines to chiral amines. This reaction is described as a two-step process where the imine is first prepared and isolated followed by hydrogenation of the isolated imine to an amine. It was found that ketones were either not hydrogenated by Ir-complexes under the same conditions or the reaction was too slow.

Hence, there is a continuing need for convenient methods for the synthesis of chiral amines from ketones.

### SUMMARY OF THE DISCLOSURE

An advantage of the present invention is the formation of chiral amines by directly and asymmetrically aminating a starting ketone.

Additional advantages, and other features of the present invention will be set forth in the description which follows and in part will become apparent to those having ordinary skill in the art upon examination of the following or may be learned from the practice of the present disclosure. The advantages may be realized and obtained as particularly pointed out in the appended claims.

According to the present invention, the foregoing and other advantages are achieved in part by a process for manufacturing a chiral amine. The process comprises admixing a ketone with an amine in the presence of a catalyst and an acid; and then exposing the admixture to a source of hydrogen to reductively aminate the ketone with the amine to form a chiral amine product. Catalysts contemplated for use in the present invention include a transition metal, such as a group VIII transition metal, that is complexed with a chiral phosphine ligand, such as f-binaphane.

Additional advantages of the present invention will become readily apparent to those skilled in this art from the following detailed description, wherein only the preferred embodiments of the present invention are shown and described, simply by way of illustration but not limitation. As will be realized, the invention is capable of other and different embodiments, and its several details are capable of modification in various obvious respects, all without departing from the spirit of the present invention. Accordingly, the drawings and description are to be regarded as illustrative in nature, and not as restrictive.

### DETAILED DESCRIPTION OF THE DISCLOSURE

The present invention stems from the discovery that chiral amines can be produced directly from ketones. After experimentation and investigation, it was discovered that reductive amination of ketones using a catalyst containing a transition metal, such as a group VIII transition metal, can be achieved, even without the isolation of intermediate compounds such as an imine. In addition, it was also determined that the presence of an acid, e.g., a Lewis acid, or acidic conditions, aids in the direct conversion of the ketone into the amine. For Ir-catalyzed asymmetric reductive amination of ketones, the presence of reducing additives, such as I₂, is also helpful for achieving high activities and enantioselectivities.

In practicing the present invention, ketones, such as aryl ketones, can be directly converted to an amine with high stereo selectivity by admixing a ketone with an amine in the presence of a transition metal catalyst complexed with a chiral phosphine ligand and an acid. The catalyst can comprise a transition metal, such as a group VIII metal, also commonly known as elements in rows 8, 9 and 10 of the Periodic Table, such as rhodium, iridium, ruthenium, palladium, etc. The transition metal, however is not limited thereto.

The catalyst used in practicing the invention includes a transition metal complexed with a chiral ligand. It is believed that any chiral phosphine ligand can be used in the present invention. There are many such ligands known in the art. In one embodiment of the present invention, the chiral phosphine ligand is selected among the following: DuPhos, BINAP, BPPM, DIPAMP, DIOP, MCCPM, BCPM, BICP, PennPhos, BPE, ChiraPhos, NorPhos, Degphos, BPPFA, JosiPhos, TRAP, TolBINAP, H8-BINAP, BINAPO, MOP, BINAPHOS, BIPHEMP, SEGPHOS, TUNAPHOS, KetalPhos, f-KetalPhos, HydroPhos, f-HydroPhos, Binaphane, f-Binaphane, Ferrotane, Walphos, Rophos, Butiphane phanephos, Madyphos, Taniaphos, Malphos, Cl-MeO-BIPHEP, BIPFUP, P-phos, JAFaphos, Spirop, MeO-BIPHEP, Bophoz and derivatives thereof. Among the chiral phosphine ligands, f-Binaphane or a derivative thereof is preferred. The f-Binaphane ligand has the structure given below.

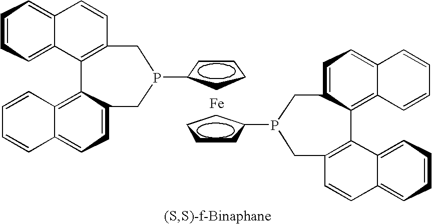

Additional information regarding the preparation of f-Binaphane and its derivatives, as well as other chiral phosphine ligands, can be found in WO 0114299, the entire disclosure of which is hereby incorporated herein by reference.

The catalyst can advantageously prepared in situ in the process of forming the chiral amine. For example, the a transition metal salt or transition metal complex can be admixed with the chiral phosphine ligand along with the other reagents. Transition metal salts or transition metal complexes include: (Rh(COD)Cl)₂; (Rh(COD)₂)X; (Ir(COD)Cl)₂; (Ir(COD)₂)X; (Ir(COD)I)₂; (Rh(NBD)Cl)₂; (Rh(NBD)₂)X; (Ir(NBD)Cl)₂; (Ir(NBD)₂)X; (Ir(NBD)I)₂; Ru(RCOO)₂(diphosphine); RuX′₂(diphosphine)(DMF)ₙ; (NH2R2){RuCl(bisphos)₂(Cl)₃}, Ru(methallyl)₂(diphosphine); Ru(aryl group)X′₂(diphosphine), RuCl₂(bisphosphine)(diamine); wherein COD is a 1,5-cycloctadiene, NBD is a norbornadiene, DMF is a dimethylformamide, R is alkyl or aryl, X is BF₄, ClO₄, SbF₆ or CF₃SO₃, X′is Cl or Br and n indicates a salvation state, e.g., an integer of 1 to 6.

In practicing one aspect of the present invention, a ketone is directly converted to a chiral amine with the aid of an acid or under acidic conditions, i.e. when the medium has a pH of less than 7. In one embodiment of the present invention, the reaction is carried out in a buffered medium having a pH of between about 3 to about 6.5, e.g., to about 4 to 6. This can be achieved by admixing a Bronsted acid with the ketone, amine and catalyst. Preferably, the medium is adjusted with a Bronsted acid to a range of between 3.5 to 6.5 with an ammonium salt or salt mixture. For example, the medium can be made acidic by admixing an ammonium salt such as NH₄X, RNH₃X, R₂NH₂X; where X is a counterion such as Cl, OTf, BF₄, etc; R is an alkyl or aryl group; and OTf is a triflate group. Additionally, the medium can be buffered with an ammonium hydroxide salt such as NH₄OH, RNH₃OH, R₂NH₂OH, where R is an alkyl or aryl group. The acidic condition can also be achieved with a mixture comprising an amine with a mild acid such as CH₃COOH, PhCOOH, HCOOH, HO₂CCO₂H, H₂CO₃, CF₃COOH or addition of NH₄OAc, and NH₄HCO₂.

As an alternative or in addition to a Bronsted acid or an acidic medium, the acid can be a Lewis acid. There are many Lewis acids that can be used in practicing certain embodiments of the present invention including titanium, zinc, aluminum, magnesium, borane, lanthanum, salts thereof and oxides thereof. For example, Lewis acids selected from the group consisting of Ti(OR)₄, TiCl₄, Zn(OTf)₂, ZnCl₂, Al(OR)₃, MgSO₄, BF₃, B(C₆F₅)₃, La(OR)₃, La(OTf)₃ and Cu(OTf)₂; wherein R is an alkyl or aryl group and OTf is a triflate group, can be used.

In another embodiment of practicing the present invention, an additive is also admixed with the ketone, amine, acid and catalyst. It is believed that the additive aids in the reduction of the intermediate imine that is likely formed in the process. There are many reducing additives that are known in the art for aiding the reduction of double bonds through hydrogenation and useful in practicing embodiments of the present invention. It has been found that iodine is preferred for reductively aminating ketones with an iridium complexed catalyst.

In practicing certain embodiments of the present invention it has been discovered that high enantioselectivity and activity can be achieved in the direct reductive amination of ketones using a transition metal complexed with f-Binaphane as a catalyst system in the presence of a Lewis acid, such as a titanium oxide, and a reducing additive, such as iodine.

To better understand the chemistry of asymmetric reduction of a ketone to a chiral amine, acetophenone 1a was chosen as a test substrate. Several other reagents were also screened including various aryl amines (aniline, benzylamine, 2,6-dimethyl aniline, o-anisidine, m-anisidine, p-anisidine) and solvents (dichloromethane, toluene, tetrahydrofuran, methanol, isopropanol), to determine optimal conditions for a working test reaction that could be studied and applied to equivalent starting ketones, amines, and other reagents. The best result, with respect to yield and enantioselectivity of chiral amine 2a (93% yield, 91% ee, and yield of imine 3a is 2%, Table 1, entry 5), was obtained with p-anisidine and dichloromethane. From experimentation, it was concluded that the Ir-f-Binaphane catalytic system is highly active and enantioselective for imine reduction. However, the formation of the imine is the limiting step in achieving complete conversion for asymmetric direct reductive amination. Table 1 shows the results for the additive effect in asymmetric direct reductive amination of acetophenone with p-anisidine.

TABLE 1^{a}

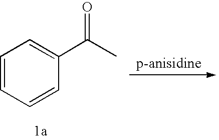

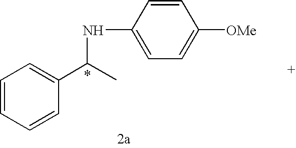

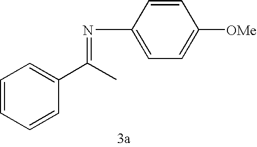

| | | Yield of | Chiral amine 2a | |
|---|---|---|---|---|
| Entry | Additive | 3a (%) | Yield (%) | e (%)^{b} |
| 1 | 10% I₂, 2.0 eq. Ti(OⁱPr)₄ | <1 | >99 | 94 |
| 2 | 10% I₂, 1.5 eq. Ti(OⁱPr)₄ | <1 | >99 | 94 |
| 3 | 10% I₂, 1.0 eq. Ti(OⁱPr)₄ | <1 | >99 | 91 |
| 4 | 10% I₂, 0.5 eq. Ti(OⁱPr)₄ | <1 | >99 | 89 |
| 5 | 10% I₂ | 2 | 93 | 91 |

| | | |
|---|---|---|
| 6 | 1.5 eq. Ti(OⁱPr)₄ | no reaction detected |

^{a}Reaction conditions: 1.2 equivalents of p-anisidine, 1 mol% Ir-(S,S)-f-Binaphane, H₂ (1000 psi), dichloromethane (DCM), room temperature, 12 h.
^{b}Absolute configurations were not determined.

Studies on the effect of reducing additives can help to optimize activities and enantioselectivities in asymmetric catalysis. In one such the investigated additive effect for the formation of imines and asymmetric on of imines is summarized in Table 1. Several Lewis acids were ed including: Ti(OⁱPr)₄, 4Å molecular sieves, MgSO₄, or TsOH. It is d that the Lewis acid tends to accelerate the formation of imines. From experimentation, Ti(OⁱPr)₄ was found to be an efficient accelerant for asymmetric reductive amination. The yield of chiral amine 2a increased from 93% (entry 5) to greater than 99% (entry 2) with the Lewis acid, and the enantioselectivity ed slightly as well (compare entries 2 and 5; 91 to 94% ee). When the amount of Ti(OⁱPr)₄ decreased from 1.5 equivalents to 0.5 equivalents, the yield of chiral amine 2a did not change, while the enantioselectivity dropped from 94 to 89% ee (entries 2 through 4). However, additional Ti(OⁱPr)₄ above 1.5 equivalents did not have any measurable improvement on enantioselectivity (compare entries 1 and 2 of Table 1).

It was also observed that iodine is a factor in the Ir-f-Binaphane catalytic system. In the presence of Ti(OⁱPr)₄ without iodine, or Ti(OⁱPr)₄ with tetrabutylamonium Iodide or acetic acid, no reaction occurs (entry 6) without iodine.

While not intending to be bound by any theory, it is believed that the amination reaction proceeds by the mechanism outlined in Scheme 1. Scheme 1 shows how the individual components of an admixture may function separately even though all of these components may be present together in a reaction stew to generate a chiral amine directly from a starting ketone. For example, in the presence of Lewis acid, such as a titanium oxide, imines were formed through an equilibrium from an intermediate, aminoalcoholatitanium (IV) complex 4. In the presence of I₂, the resulting imine was hydrogenated by the Ir-f-Binaphane complex to yield the chiral amine. A similar intermediate 4 was reported by Bhattacharyya et al in an other reductive amination reaction.

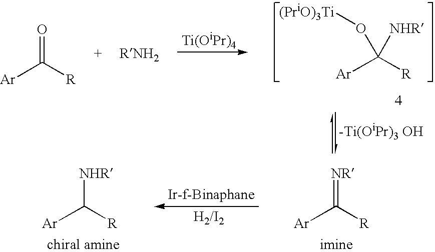

Scheme 1. The proposed mechanism of asymmetric reductive amination in the presence of Ti(OⁱPr)₄, and I₂.

Under the optimized conditions noted in Table 1, a series of aryl ketones 1a-k were subjected to direct amination (Table 2). Complete conversions were achieved for all substrates illustrating the versatility of directly aminating ketones in accordance with embodiments of the present invention. The simplest aryl ketone 1a was reductively aminated with 94% ee using an Ir-f-Binaphane complex as the catalyst (entry 1). This result is superior to enatiomeric excess' obtained with other phosphine ligands (entry 12, 16% ee with (R)-BINAP; entry 13, 25% ee with (R)-BIPHEP). When the alkyl (R) group of the ketone was changed from Me to Et and then ⁿBu, the ee value dropped from 94 to 85 and then 79%, respectively (entries 1-3). The drop in enantiomeric excess is likely due to the steric hindrance by an increasingly bulky substituent.

The electronic effects of the substrate were also examined with a series of substituted acetophenones (entries 4-10). An electron-donating para substituent on the acetophenone was found to give higher enantioselectivities (compare entry 6, 96% ee, to entry 5). High enantioselectivity, i.e. greater than about 60% ee, was also achieved on reductive amination of a heterocyclic ketone (entry 11, 92% ee). Unfortunately, the Ir-f-Binaphane catalytic system was not optimized for asymmetric direct reductive amination of alkyl ketones. Table 2 shows the results of asymmetric direct reductive amination of various aryl ketones with p-anisidine.

TABLE 2^{a}

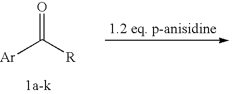

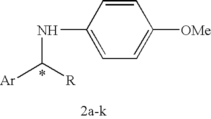

| Entry | Substrate | Ar | R | ee (%) | Configuration |
|---|---|---|---|---|---|
| 1 | 1a^{b} | Ph | Me | 94 | R−(+)^{e} |
| 2 | 1b^{b} | Ph | Et | 85 | (+)^{f} |
| 3 | 1c^{b} | Ph | ⁿBu | 79 | R−(+)^{e} |
| 4 | 1d^{b} | 2-Me—C₆H₄ | Me | 44 | (+)^{f} |
| 5 | 1e^{b} | 3-Me—C₆H₄ | Me | 89 | (+)^{f} |
| 6 | 1f^{b} | 4-Me—C₆H₄ | Me | 96 | (+)^{f} |
| 7 | 1g^{b} | 4-OMe—C₆H₄ | Me | 95 | (+)^{f} |
| 8 | 1h^{b} | 4-F—C₆H₄ | Me | 93 | (−)^{f} |
| 9 | 1i^{b} | 4-Cl—C₆H₄ | Me | 92 | (+)^{f} |
| 10 | 1j^{b} | 4-Br—C₆H₄ | Me | 94 | (+)^{f} |
| 11 | 1k^{b} | 2-furan | Me | 92 | (+)^{f} |
| 12 | 1a^{c} | Ph | Me | 16 | S−(−)^{e} |
| 13 | 1a^{d} | Ph | Me | 25 | S−(−)^{e} |

^{a}Reaction conditions: 1 mol% Ir-chiral ligand, H₂ (1000 psi), DCM, 10% I₂, 1.5 eq. Ti(OⁱPr)₄, room temperature, 12 h.
^{b}Chiral ligand: (S,S)-f-Binaphane.
^{c}Chiral ligand: (R)-BINAP.
^{d}Chiral ligand: (R)-MeO-BIPHEP.
^{e}Absolute configurations were determined by the sign of optical rotation.
^{f}Absolute configurations were not determined.

It is noteworthy that the N-p-methoxyphenyl group on the chiral amine 2, can be easily removed by oxidation with CAN (cerium ammonium nitrate). Based on this strategy, the chiral primary amine 5 was synthesized from acetophenone through a two-step asymmetric direct reductive amination process

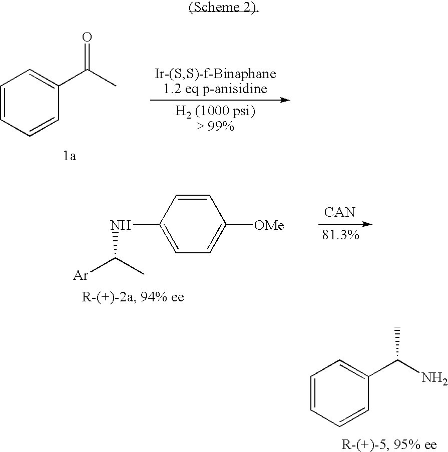

Scheme 2. Simple and efficient synthesis of a chiral primary amine.

In another aspect of the present invention, primary amine chiral compounds can be formed by admixing a ketone, e.g., an aryl ketone, with an amine having a leaving group. The admixture can also contain a Lewis acid, such as a titanium oxide, a reducing additive, such as iodine, and a catalyst, which comprises a transition metal complexed with a chiral phosphine ligand such as f-Binaphane. Exposing this admixture to a source of hydrogen, reductively aminates the aryl ketone with the amine having the leaving group to form a chiral compound having the amine with the leaving group. Removing the leaving group from the amine can result in a primary amine chiral compound depending on the substitution of the starting amine.

The Ir-f-Binaphane complex shows high activities and enantioselectivities (up to 96% ee) for asymmetric direct reductive amination of aryl ketones in the presence of Ti(OⁱPr)₄ and I₂. This system can also be practiced with a variety of substituted amines, including amines having readily liable leaving groups. Hence, a simple and efficient two-step process for preparation of chiral primary amines can also be practiced with these reagents.

### EXPERIMENTAL

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

General procedure for asymmetric direct reductive amination: In a glovebox that was filled with N₂, Aryl ketones (0.5 mmol), p-anisidine (74 mg, 0.6 mmol), titanium(IV) isopropoxide (213 mg, 0.75 mmol), and iodine (13 mg, 0.05 mmol) were dissolved in 2 ml of DCM. The Ir-(S,S)-f-Binaphane complex was made in situ by mixing [Ir(COD)Cl]₂ (1.7 mg, 0.0025 mmol) and (S,S)-f-Binaphane (4.4 mg, 0.0055 mmol) in 3 ml of DCM. The mixture was stirred for 30 min, and transferred to the substrate solution. This reaction solution was transferred to a Parr bomb. The direct reductive amination was performed at room temperature under 1000 psi of hydrogen for 12 hours. After the reaction was finished, hydrogen was released carefully and the reaction mixture was passed through a silica gel plug eluted with EtOAc/hexane in a 6:1 ratio. The solvent was removed under vacuum to yield the product as yellow oil. The enantiomeric excess was measured by using HPLC with a chiral column without further purification. The absolute configurations were determined by the sign of the optical rotation. The following chiral amines were prepared in accordance with the foregoing procedure.

R-(+)-4-Methoxy-N-(1-phenyl-ethyl) aniline 2a: [α]²⁵_{D}=+7.0° (c=2, CHCl₃); ¹H-NMR (360 MHz, CDCl₃) δ 1.44 (3H, d, J=6.72 Hz, —CH₃), 3.63 (3H, s, O—CH₃), 3.74 (1H, br., NH), 4.37 (1H, q, J=6.67 Hz, —CH), 6.42-6.45 (2H, m, Ar—H), 6.63-6.68 (2H, m, Ar—H), 7.17-7.20 (1H, m, Ar—H), 7.25-7.33 (4H, m, Ar—H) ppm; ¹³C-NMR (90.6 MHz, CDCl₃) δ 25.3, 54.5, 55.9, 114.8, 115.0, 126.1, 127.0, 128.8, 141.8, 145.7, 152.1 ppm; HRMS (ADCI) calcd. for C₁₅H₁₈NO [M+H]⁺ 228.1388, found 228.1424; 94% ee by HPLC ( Chiralcel OD, hexane:2-propanol=97:3, 1.0 ml/min, R isomer t₁=11.8 min, S isomer t₂=13.2 min).

(+)-N-(4-Methoxyphenyl)-1-phenylpropylamine 2b: [α]²⁵_{D}=+24.6° (c=2, CHCl₃); ¹H-NMR (360 MHz, CDCl₃) δ 1.27(3H, t, —CH₃), 2.09-2.18 (2H, m, —CH₂), 4.00 (3H, s, O—CH₃), 4.16 ( 1H, br., NH), 4.49(1H, t, —CH), 6.78-6.83 (2H, m, Ar—H), 7.00-7.04 (2H, m, Ar—H), 7.52-7.56 (1H, m, Ar—H), 7.61-7.67 (4H, m, Ar—H) ppm; ¹³C-NMR (90.6 MHz, CDCl₃) δ 11.0, 31.8, 55.9, 60.7, 114.6, 114.9, 126.7, 127.0, 128.6, 142.0, 144.3, 152.0 ppm; HRMS (ADCI) calcd. for C₁₆H₂₀NO [M+H]⁺ 242.1545, found 242.1535; 85% ee by HPLC ( Chiralcel OD, hexane:2-propanol=97:3, 1.0 ml/min, (+)-isomer t₁=8.9 min, (−)-isomer t₂=9.8 min.

R-(+)-N-(4-Methoxyphenyl)-(1-phenyl-pentyl)-amine 2c: [α]²⁵_{D}=+5.7° (c=2, CHCl₃); ¹H-NMR (360 MHz, CDCl₃) δ 1.14 (3H, t, —CH₃), 1.54-1.64(4H, m, CH₂), 1.97-2.06 (2H, m, CH₂), 3.91 (3H, s, O—CH₃), 3.93 (1H, br., NH), 4.47 (1H, t, —CH), 6.69-6.74 (2H, m, Ar—H), 6.91-6.95 (2H, m, Ar—H), 7.43-7.48 (1H, m, Ar—H), 7.52-7.60 (4H, m, Ar—H) ppm; ¹³C-NMR (90.6 MHz, CDCl₃) δ 14.1, 22.8, 28.7, 38.9, 55.9, 59.2, 114.6, 114.9, 126.6, 126.9, 128.6, 142.0, 144.8, 152.0 ppm; HRMS (ADCI) calcd. for C₁₈H₂₄NO [M+H]⁺ 270.1858, found 270.1843; 79% ee by HPLC ( Chiralcel OD, hexane:2-propanol=97:3, 1.0 ml/min, R isomer t₁=7.7 min, S isomer t₂=8.3 min).

(+)-N-(4-Methoxyphenyl)-(1-o-tolyl-ethyl)-amine 2d: [α]²⁵_{D}=+9.2° (c=2, CHCl₃); ¹H-NMR (360 MHz, CDCl₃) δ 1.67 (3H, d, J=6.60 Hz, —CH₃), 2.66 (3H, s, Ar—CH₃), 3.90 (3H, s, O—CH₃), 3.91 (1H, br., NH), 4.83 (1H, q, J=6.53 Hz, —CH), 6.62-6.65 (2H, m, Ar—H), 6.89-6.93 (2H, m, Ar—H), 7.35-7.41 (3H, m, Ar—H), 7.64-7.66 (1H, m, Ar—H) ppm; ¹³C-NMR (90.6 MHz, CDCl₃) δ 19.1, 23.2, 50.6, 55.8, 114.4, 115.0, 124.8, 126.7, 130.7, 134.7, 141.7, 143.1, 152.0 ppm; HRMS (ADCI) calcd. for C₁₆H₂₀NO [M+H]⁺ 242.1545, found 242.1555; 44% ee by HPLC ( Chiralcel OD, hexane:2-propanol=97:3, 1.0 ml/min, (+)-isomer t₁=9.1 min, (−)-isomer t₂=11.8 min).

(+)-N-(4-Methoxyphenyl)-(1-m-tolyl-ethyl)-amine 2e: [α]²⁵_{D}=+8.1° (c=2, CHCl₃); ¹H-NMR (360 MHz, CDCl₃) δ 1.82 (3H, d, J=6.67 Hz, —CH₃), 2.68(3H, s, Ar—CH₃), 4.02 (3H, s, O—CH₃), 4.04 (1H, br., NH), 4.72 (1H, q, J=6.65 Hz, —CH), 6.81-6.84 (2H, m, Ar—H), 7.03-7.06 (2H, m, Ar—H), 7.37-7.39 (1H, m, Ar—H), 7.50-7.57(3H, m, Ar—H) ppm; ¹³C-NMR (90.6 MHz, CDCl₃) δ 21.6, 25.2, 54.4, 55.8, 114.7, 114.9, 123.1, 126.7, 127.7, 128.6, 138.2, 141.9, 145.7, 152.0 ppm; HRMS (ADCI) calcd. for C₁₆H₂₀NO [M+H]⁺ 242.1545, found 242.1544; 89% ee by HPLC ( Chiralcel OD, hexane:2-propanol=97:3, 1.0 ml/min, (+)-isomer t₁=10.0 min, (−)-isomer t₂=11.5 min).

(+)-N-(4-Methoxyphenyl)-(1-p-tolyl-ethyl)-amine 2f: [α]²⁵_{D}=+10.7° (c=2, CHCl₃); ¹H-NMR (360 MHz, CDCl₃) δ 1.76 (3H, d, J=6.69 Hz, —CH₃), 2.61(3H, s, Ar—CH₃), 3.96 (3H, s, O—CH₃), 3.97 (1H, br., NH), 4.68(1H, q, J=6.63 Hz, —CH), 6.75-6.79 (2H, m, Ar—H), 6.97-7.01 (2H, m, Ar—H), 7.40-7.45 (2H, m, Ar—H), 7.53-7.55(2H, m, Ar—H) ppm; ¹³C-NMR (90.6 MHz, CDCl₃) δ 21.2, 25.2, 54.0, 55.8, 114.9, 125.9, 129.4, 136.4, 141.8, 142.6, 152.0 ppm; HRMS (ADCI) calcd. for C₁₆H₂₀NO [M+H]⁺ 242.1545, found 242.1550; 96% ee by HPLC (Chiralcel OD, hexane:2-propanol=97:3, 1.0 ml/min, (+)-isomer t₁=9.8 min, (−)-isomer t₂=11.2 min).

(+)-N-(4-Methoxyphenyl)-{1-(p-methoxy-phenyl)-ethyl}-amine 2g: [α]²⁵_{D}=+23.8° (c=2, CHCl₃); ¹H-NMR (360 MHz, CDCl₃) δ 1.78 (3H, d, J=6.67 Hz, —CH₃), 3.99(3H, s, O—CH₃), 4.03 (1H, br., NH), 4.07 (3H, s, O—CH₃), 4.69(1H, q, J=6.61 Hz, —CH), 6.78-6.81 (2H, m, Ar—H), 7.00-7.03 (2H, m, Ar—H), 7.15-7.23 (2H, m, Ar—H), 7.59(2H, d, J=8.65 Hz, Ar—H) ppm; ¹³C-NMR (90.6 MHz, CDCl₃) δ 25.1, 53.7, 55.3, 55.8, 114.1, 114.7, 114.9, 127.0, 137.6, 141.8, 152.0, 158.6 ppm; HRMS (ADCI) calcd. for C₁₆H₂₀NO₂ [M+H]⁺ 258.1494, found 258.1498; 95% ee by HPLC ( Chiralcel OD, hexane:2-propanol=97:3, 1.0 ml/min, (+)-isomer t₁=14.5 min, (−)-isomer t₂=16.3 min).

(−)-N-(4-Methoxyphenyl)-{1-(p-fluoro-phenyl)-ethyl}-amine 2h: [α]²⁵_{D}=−6.7° (c=2, CHCl₃); ¹H-NMR (360 MHz, CDCl₃) δ 1.73 (3H, d, J=6.66 Hz, —CH₃), 3.90 (1H, br., NH), 3.96 (3H, s, O—CH₃), 4.66 (1H, q, J=6.62 Hz, —CH), 6.71-6.74 (2H, m, Ar—H), 6.97-6.99 (2H, m, Ar—H), 7.24-7.29 (2H, m, Ar—H), 7.57-7.61 (2H, m, Ar—H) ppm; ¹³C-NMR (90.6 MHz, CDCl₃) δ 25.3, 53.8, 55.8, 114.9, 115.4, 115.6, 127.5, 127.6, 141.3, 141.4, 141.5, 152.2, 160.5, 163.2 ppm; HRMS (ADCI) calcd. for C₁₅H₁₇NOF [M+H]⁺ 246.1294, found 246.1296; 93% ee by HPLC ( Chiralcel OD, hexane:2-propanol=97:3, 1.0 ml/min, (−)-isomer t₁=14.2 min, (+)-isomer t₂=16.5 min).

(+)-N-(4-Methoxyphenyl)-{1-(p-chloro-phenyl)-ethyl}-amine 2i: [α]²⁵_{D}=+23.1° (c=2, CHCl₃); ¹H-NMR (360 MHz, CDCl₃) δ 1.55 (3H, d, J=6.71 Hz, —CH₃), 3.78 (3H, s, O—CH₃), 3.82 (1H, br., NH), 4.47 (1H, q, J=6.69 Hz, —CH), 6.53-6.56 (2H, m, Ar—H), 6.79-6.82 (2H, m, Ar—H), 7.35-7.40 (4H, m, Ar—H) ppm; ¹³C-NMR (90.6 MHz, CDCl₃) δ 25.2, 53.8, 55.8, 114.9, 127.4, 128.9, 132.4, 141.4, 144.2, 152.2 ppm; HRMS (ADCI) calcd. for C₁₅H₁₇NOCl [M+H]⁺ 262.0999, found 262.0990; 92% ee by HPLC ( Chiralcel OD, hexane:2-propanol=97:3, 1.0 ml/min, (+)-isomer t₁=15.4 min, (−)-isomer t₂=18.7 min).

(+)-N-(4-Methoxyphenyl)-{1-(p-bromo-phenyl)-ethyl}-amine 2j: [α]²⁵_{D}=+28.4° (c=2, CHCl₃); ¹H-NMR (360 MHz, CDCl₃) δ 1.52 (3H, d, J=(6.72 Hz, —CH₃), 3.75 (3H, s, O—CH₃), 3.78 (1H, br., NH), 4.42 (1H, q, J=6.67 Hz, —CH), 6.48-6.53 (2H, m, Ar—H), 6.75-6.79 (2H, m, Ar—H), 7.27-7.31 (2H, m, Ar—H), 7.47-7.51 (2H, m, Ar—H) ppm; ¹³C-NMR (90.6 MHz, CDCl₃) δ 25.2, 53.9, 55.8, 114.7, 114.9, 120.5, 127.8, 131.8, 141.3, 144.8, 152.2 ppm; HRMS (ADCI) calcd. for C₁₅H₁₇NOBr [M+H]⁺ 306.0494, found 306.0505; 94% ee by HPLC (Chiralcel OD, hexane:2-propanol=97:3, 1.0 ml/min, (+)-isomer t₁=16.5 min, (−)-isomer t₂=20.2 min).

(+)-N-(4-Methoxyphenyl)-{1-(2′-furan)-ethyl}-amine 2k: [α]²⁵_{D}=+92.9° (c=2, CHCl₃); ¹H-NMR (360 MHz, CDCl₃) δ 1.79 (3H, d, J=6.72 Hz, —CH₃), 3.80 (1H, br., NH), 3.98 (3H, s, O—CH₃), 4.81 (1H, q, J=6.71 Hz, —CH), 6.39-6.40(1H, m, Ar—H), 6.53-6.54 (1H, m, Ar—H), 6.83-6.88 (2H, m, Ar—H), 6.99-7.04 (2H, m, Ar—H), 7.58-7.59 (1H, m, Ar—H) ppm; ¹³C-NMR (90.6 MHz, CDCl₃) δ 21.1, 48.5, 55.8, 105.1, 105.3, 110.1, 110.3, 114.9, 115.3, 141.3, 141.5, 152.6, 157.7 ppm; HRMS (ADCI) calcd. for C₁₃H₁₆NO₂ [M+H]⁺ 218.1181, found 218.1212; 92% ee by HPLC (Chiralpak OJ, hexane:2-propanol=80:20, 1.0 ml/min, (−)-isomer t₁=12.6 min, (+)-isomer t₂=16.1 min).

Procedure for oxidation deprotection of chiral amine 2a: The chiral amine, R-(+)-4-Methoxy-N-(1-phenyl-ethyl) aniline 2a (85 mg, 0.395 mmol, 93.3% ee) was dissolved into a mixture of MeOH/H₂O (20 ml, 4:1), after the reaction solution was cooled to 0° C., CAN (cerium ammonium nitrate) ( 866 mg, 1.58 mmol) was added in one portion, the resulting reaction system was stirred for 6 hours at the same temperature. Water was added and the solution was washed with CH₂Cl₂. The aqueous solution was made alkaline by adding 1N NaOH, and then extracted with ethyl acetate. The combined organic solution was washed with brine and dried over Na₂SO₄. The solvent was removed under vacuum, and the residue was purified through silica gel column chromatography, eluted with ethyl acetate, to afford the chiral primary amine 5 as yellow oil (39 mg, 81.3% yield).

R-(+)-a-Methyl-benzylamine 5: [α]²⁵_{D}=+29.9° (c=1, CHCl₃); ¹H-NMR (360 MHz, CDCl₃) δ 1.55 (3H, d, J=6.59 Hz, —CH₃), 1.63 ( 2H, br., NH2), 4.27 (1H, q, J=6.57 Hz, —CH), 7.39-7.42(1H, m, Ar—H), 7.47-7.53 (4H, m, Ar—H) ppm; ¹³C-NMR (90.6 MHz, CDCl₃) δ 25.6, 51.1, 125.5, 126.6, 128.3, 147.7 ppm. 95% ee by GC after protection with acetic anhydride (Chiralselect 1000, dimension 30 m×0.25 mm, column temperature 160° C., carrier gas: He (1 ml/min), S-isomer t₁=18.7 min, R-isomer t₂=19.7 min).

Only the preferred embodiment of the present invention and examples of its versatility are shown and described in the present disclosure. It is to be understood that the present invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A process for manufacturing a chiral amine, the process comprising:
admixing (i) a ketone, (ii) an amine, (iii) an acid, and (iv) a catalyst, which comprises a transition metal complexed with a chiral phosphine ligand; and
exposing the admixture to a source of hydrogen to reductively aminate the ketone with the amine to form a chiral amine product.

2. The process of claim 1, wherein the acid is a Lewis acid.

3. The process of claim 1, wherein the ketone, amine, acid, and catalyst are in a medium buffered to a pH of about 3.5 to about 6.5.

4. The process of claim 1, wherein the transition metal is selected from the group consisting of rhodium, iridium, ruthenium, palladium and combinations thereof.

5. The process of claim 2, wherein the Lewis acid is selected from the group consisting of: Ti(OR)₄, TiCl₄, Zn(OTf)₂, ZnCl₂, Al(OR)₃, MgSO₄, BF₃, B(C₆F₅)₃, La(OR)₃, La(OTf)₃ and Cu(OTf)₂; wherein R is an alkyl or aryl group and OTf is a triflate group.

6. The process of claim 1, wherein the transition metal is in the form of a salt or complex selected from the group consisting of: (Rh(COD)Cl)₂; (Rh(COD)₂)X; (Ir(COD)Cl)₂; (Ir(COD)₂)X; (Ir(COD)I)₂; (Rh(NBD)Cl)₂; (Rh(NBD)₂)X; (Ir(NBD)Cl)₂; (Ir(NBD)₂)X; (Ir(NBD)I)₂; Ru(RCOO)₂(diphosphine); RuX′₂(diphosphine)(DMF)ₙ; (NH2R2){RuCl(bisphos)₂ (Cl)₃}, Ru(methallyl)₂(diphosphine); Ru(aryl group)X′₂(diphosphine), RuCl₂(bisphosphine)(diamine); wherein COD is a 1,5-cycloctadiene, NBD is a norbornadiene, DMF is a dimethylformamide, R is alkyl or aryl, X is BF₄, ClO₄, SbF₆ or CF₃SO₃, X′ is Cl or Br and n indicates a salvation state.

7. The process of claim 4, wherein the diphosphine is selected from the group consisting of: DuPhos, BINAP, BPPM, DIPAMP, DIOP, MCCPM, BCPM, BICP, PennPhos, BPE, ChiraPhos, NorPhos, Degphos, BPPFA, JosiPhos, TRAP, TolBINAP, H8-BINAP, BINAPO, MOP, BINAPHOS, BIPHEMP, SEGPHOS, TUNAPHOS, KetalPhos, f-KetalPhos, HydroPhos, f-HydroPhos, Binaphane, f-Binaphane, Ferrotane, Walphos, Rophos, Butiphane phanephos, Madyphos, Taniaphos, Malphos, Cl-MeO-BIPHEP, BIPFUP, P-phos, JAFaphos, Spirop, MeO-BIPHEP, and Bophoz.

8. The process of claim 1, wherein the chiral phosphine ligand is f-Binaphane.

9. The process of claim 1, wherein the catalyst is formed in situ by mixing a transition metal salt or transition metal complex with the chiral phosphine ligand.

10. A process for manufacturing a chiral aryl amine, the process comprising:
admixing (i) an aryl ketone, (ii) an amine, (iii) an acid, and (iv) a catalyst, which comprises a transition metal complexed with a chiral phosphine ligand; and
exposing the admixture to a source of hydrogen to reductively aminate the aryl ketone with the amine to form a chiral aryl amine product.

11. The process of claim 10, wherein the transition metal is a group VIII transition metal and combinations thereof.

12. The process of claim 10, wherein the acid is selected from the group consisting of: Ti(OR)₄, TiCl₄, Zn(OTf)₂, ZnCl₂, Al(OR)₃, MgSO₄, BF₃, B(C₆F₅)₃, La(OR)₃, La(OTf)₃ and Cu(OTf)₂; wherein R is an alkyl or aryl group and OTf is a triflate group.

13. The process of claim 10, further admixing iodine with the aryl ketone, amine, acid, and catalyst prior to exposing the admixture to the source of hydrogen.

14. The process of claim 10, wherein the chiral phosphine ligand is selected from the group consisting of: DuPhos, BINAP, BPPM, DIPAMP, DIOP, MCCPM, BCPM, BICP, PennPhos, BPE, ChiraPhos, NorPhos, Degphos, BPPFA, JosiPhos, TRAP, TolBINAP, H8-BINAP, BINAPO, MOP, BINAPHOS, BIPHEMP, SEGPHOS, TUNAPHOS, KetalPhos, f-KetalPhos, HydroPhos, f-HydroPhos, Binaphane, f-Binaphane, Ferrotane, Walphos, Rophos, Butiphane phanephos, Madyphos, Taniaphos, Malphos, Cl-MeO-BIPHEP, BIPFUP, P-phos, JAFaphos, Spirop, MeO-BIPHEP, and Bophoz.

15. A process for manufacturing a primary amine chiral compound, the process comprising:
admixing (i) an aryl ketone, (ii) an amine having a leaving group, (iii) a Lewis acid, and (iv) a catalyst, which comprises a transition metal complexed with a chiral phosphine ligand;
exposing the admixture to a source of hydrogen to reductively aminate the aryl ketone with the amine having the leaving group to form a chiral compound having the amine with the leaving group; and
removing the leaving group from the amine to form a primary amine chiral compound.

16. The process of claim 15, further admixing iodine with the aryl ketone, amine, acid, and catalyst prior to exposing the admixture to the source of hydrogen.

17. The process of claim 16, wherein the chiral phosphine ligand is f-Binaphane.

18. A composition comprising (i) an aryl ketone, (ii) an amine, (iii) a Lewis acid, (iv) iodine, and (v) a catalyst, which comprises a group VIII transition metal complexed with a chiral phosphine ligand.

19. The composition of claim 18 wherein the chiral phosphine ligand is f-Binaphane.
